(19) United States
(12) Patent Application Publication
(10) Pub. No.: US 2013/0053968 A1
(43) Pub. Date: Feb. 28, 2013

(54) Patient Specific Joint Prosthesis

(71) Applicants: Reto NARDINI, Oberdorf (CH); Adrian Glauser, Solothurn (CH); Jean-Pierre Kassi, Solothurn (CH)

(72) Inventors: Reto NARDINI, Oberdorf (CH); Adrian Glauser, Solothurn (CH); Jean-Pierre Kassi, Solothurn (CH)

(21) Appl. No.: 13/401,520

(22) Filed: Feb. 21, 2012

Related U.S. Application Data
(60) Provisional application No. 61/475,061, filed on Apr. 13, 2011.

(51) Int. Cl. A61F 2/40

(52) U.S. Cl. 623/19.11

(57) ABSTRACT

A patient-specific implant includes (a) a body having a convex exterior surface configured to attach to a bone and a concave interior surface configured to receive a head portion of another bone, the body including a plurality of holes extending therethrough, the holes sized and shaped to receive a bone fixation element; and (b) a gap filler formed on the exterior surface of the body and having dimensions and a position corresponding to a gap between the exterior surface of the body and the bone to which the implant is to be attached.

### PRIORITY CLAIM

The present application claims priority to U.S. Provisional Application Ser. No. 61/475,061 filed on Apr. 13, 2011 and entitled “Patient Specific Joint Prosthesis,” the entire disclosure of which is incorporated herein by reference.

### FIELD OF THE INVENTION

The present invention relates to a device for treating a bone and, in particular, relates to an implant for treating a joint.

### BACKGROUND

The shoulder joint may become damaged via, for example, arthritis or a fracture, which may require a joint replacement. A shoulder joint replacement prosthesis may include a socket implant fixed to the scapula and a ball shaped implant attached to the humerus. The socket implant may be fixed to the scapula using a plurality of bone fixation elements inserted through portions of the scapula.

Known socket implants have been manufactured in standard implant sizes and shapes. Although it is ideal for an exterior surface of the socket implant to contact the glenoid, coracoid process and acromion portions of the scapula, contact with one of these three portions of the scapula may need to be sacrificed to ensure a desired orientation of a standard size socket implant in a given patient's anatomy. Thus, the bone fixation elements fixing the socket implant to the scapula carry all of the load. If the bone fixation elements fail, the entire prosthesis will fail requiring an additional corrective surgery.

### SUMMARY OF THE INVENTION

The present invention relates to a patient-specific implant which includes a body having a convex exterior surface configured to attach to a bone and a concave interior surface configured to receive a head portion of another bone, the body including a plurality of holes extending therethrough, the holes sized and shaped to receive a bone fixation element and a gap filler formed on the exterior surface of the body and having dimensions and a position corresponding to a gap between the exterior surface of the body and the bone to which the implant is to be attached.

### BRIEF DESCRIPTION OF THE DRAWINGS

FIG. 1 shows a perspective view of an implant according to an exemplary embodiment of the present invention;

FIG. 2 shows another perspective view of the implant of FIG. 1;

FIG. 3 shows yet another perspective view of the implant of FIG. 1;

FIG. 4 shows a perspective view of an implant according to an alternate exemplary embodiment of the present invention; and

FIG. 5 shows a perspective view of an implant according to a further exemplary embodiment of the present invention.

### DETAILED DESCRIPTION

The present invention may be further understood with reference to the following description and the appended drawings, wherein like elements are referred to with the same reference numerals. The present invention relates to a device for treating a bone and, in particular, relates to an implant for treating a joint. The exemplary embodiments describe a patient specific socket implant including gap fillers formed thereon to correspond specifically to a patient's anatomy. Although exemplary embodiments of the present invention specifically describe an implant for treating a shoulder joint, it will be understood by those of skill in the art that the present invention may be used to treat any ball and socket joint of the body.

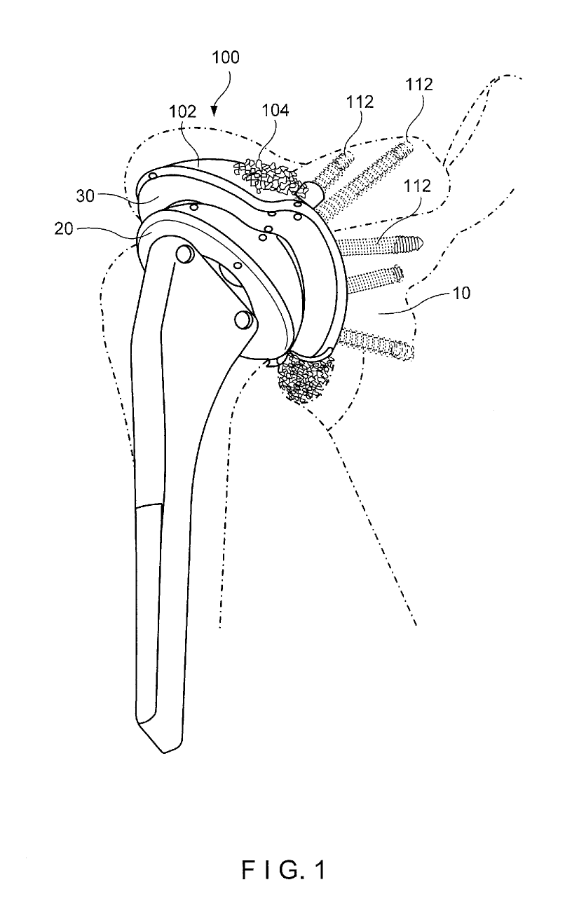

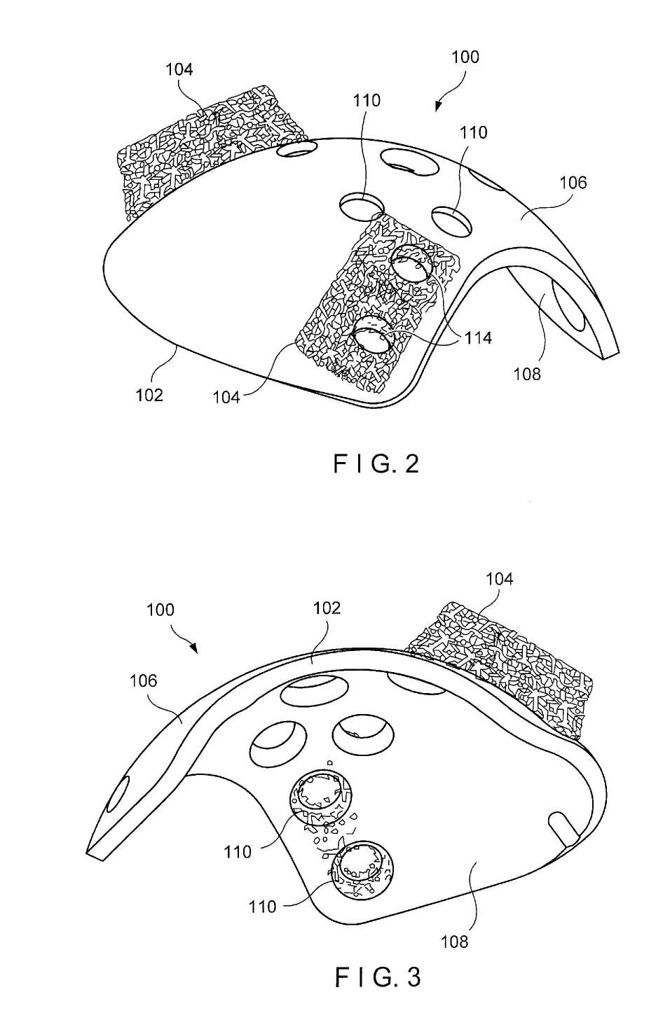

As shown in FIGS. 1-3, an implant 100 according to an exemplary embodiment of the present invention comprises a body 102 and a patient-specific gap filler 104 formed thereon to fill a gap between an exterior, bone contacting surface 106 of the body 102 and portions of a bone 10 (e.g., the scapula) on which the implant 100 is to be fixed. The implant 100 according to this embodiment is particularly suited to replace and provide support to a socket portion of a ball and socket joint such as, for example, the glenoid fossa of a shoulder joint. The exterior surface 106 is substantially convex and is configured for attachment to a socket portion of a joint such as, for example, a scapula (e.g., shoulder blade). The interior surface 108 is substantially concave and sized and shaped to receive a ball portion of the joint such as, for example, a humeral head, or a prosthetic humeral head 20. The interior surface 108 may also be configured to be attached to an interfacing layer 30, which includes a smooth surface for interfacing with the ball portion 20 of the joint.

The body 102 includes a plurality of holes 110 extending therethrough, with each of the holes 110 sized and shaped to receive a bone fixation element 112 therethrough for fixing the socket implant 100 to the bone. Each of the holes 110 is positioned along a portion of the body 102 corresponding to a desired point of fixation to the bone 10. For example, the holes 110 may be positioned along the body 102 in positions corresponding to points of contact with the glenoid, coracoid process and acromion of the scapula. The body 102 may be formed of any biocompatible material suitable for implantation in a living body and having the strength and other physical characteristics required for known implants. For example, the body 102 may be formed of titanium, titanium alloys (e.g., Ti6A14V, Ti6A17N, TiCP) or stainless steel alloys (e.g., 316L).

The gap filler 104 may be added to the exterior surface 106 of the body 102 using additive machining such that a standard body 102 which did not previously include any gap fillers 104 may be altered to suit a patient's specific anatomy. As would be understood by those skilled in the art, a patient's anatomy may be determined by imaging the bone 10 via, for example, a CT scan or an X-ray, and using a template of the body 102 to determine the required dimensions of one or more gap fillers 104 required to fill gaps which would exist between the exterior surface 106 of the body 102 and a surface of the bone 10 if the body 102 were mounted without gap fillers 104. In one exemplary embodiment, the template of the body 102 may be a 2D template positioned over a portion of the image corresponding to a position along the bone 10 on which the body 102 will be fixed. Gaps between an exterior surface of the template of the body 102 may be filled with templates of the gap fillers 104, which may be selected from a variety of 2D templates of varying sizes and shapes. The templates of the body 102 and the gap fillers 104 may be used to determine dimensions of the body 102 and the gap fillers 104 of the implant 100. In another embodiment, the user may use a 3D image of the bone 10 to obtain a data set defining dimensions of a digitalized 3D template for the implant 100. The 3D template may be used to determine dimensions of the body 102 and the gap fillers 104. The dimensions of the gap filler 104 to be added to the body 102 may be determined by, for example, a surgeon, using the templates as described above, or an engineer using the 3D data set of the imaged bone 10.

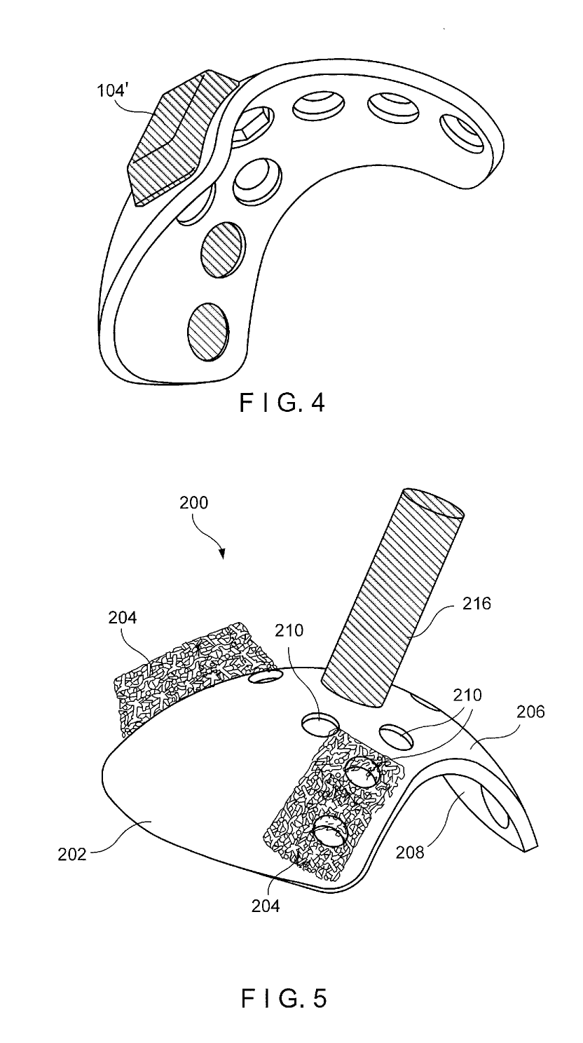

The gap filler 104 may extend over one or more of the holes 110 and may include openings 114 sized and shaped to correspond to and align with the holes 110 over which the gap filler 104 extends. The gap filler 104 may be formed of a porous material facilitating bone growth thereinto—i.e., permitting bone to grow into interstitial spaces formed by pores in the gap filler 104. For example, the gap filler 104 may be formed of a mesh-like material. As would be understood by those skilled in the art, additive machining maybe used to generate any porous structure specified by the user using any of a variety of metal materials such as, for example, titanium alloys or stainless steel alloys. It may be desired to form the gap filler 104 with a porous structure having dimensions similar to a human trabecular bone to facilitate bone ingrowth. For example, the gap filler 104 may have pores ranging in size from 0.1 mm to 1.0 mm. Alternatively, as shown in FIG. 4, a gap filler 104′ may be formed of a solid material. The gap filler 104′ may be formed of the same material as the body 102. The implant 100 may include any number of gap fillers 104 so long as the gap fillers 104 fill gaps between the exterior surface 106 and the bone 10.

As shown in FIG. 5, an implant 200 according to a further embodiment may be substantially similar to the implant 100 but further comprises an elongated element 216 providing an increased fixation stability of the implant 200 for situations in which a portion of the bone (e.g., the scapula) onto which the implant 200 is to be fixed has previously been removed (e.g., in an earlier surgery). Similarly to the implant 100, the implant 200 comprises a body 202 which may be modified by a user to include one or more gap fillers 204 as desired to conform an exterior surface thereof to a shape of corresponding portions of the patient's anatomy. The body 202 has a substantially convex exterior surface 206 for attachment to the bone, a substantially concave 208 interior surface and a plurality of holes 210 sized and shaped to receive bone fixation elements therethrough. The elongated element 216 may be similarly formed on the exterior surface 206 via, for example, an additive machining process and may be shaped to substantially match the void in the bone. For example, a substantially cylindrical elongated element 216 may be formed for insertion into an opening or groove which was formed in the bone during an earlier surgical procedure. For example, such an opening may have been reamed through the bone to accommodate portions of an implant which has been subsequently removed. It will be understood by those of skill in the art, however, that the elongated element 216 may be any of a variety of shapes so long as the elongated element 216 is sized and shaped for insertion into an opening formed in the bone. Similarly to the gap fillers 104, 204, the elongated element 216 may be formed of a porous material to encourage bone in-growth or, alternatively, may be formed in a solid configuration.

According to an exemplary method for forming the patient-specific implant 100 of the present invention, a surgeon or other professional images the bone 10 on which the implant 100 is to be fixed via, for example, a CT scan and positions a template of the implant 100 on the imaged bone 10 in a desired orientation, to determine the number and the dimensions of the gap fillers 104 required to be added to the body 102 to conform its shape to the patient's anatomy. For example, the dimensions and positions of each gap filler 104 may be determined to fill a gap between a portion of the exterior surface 106 of the body 102 and one of the glenoid, coracoid process and the acromion of a scapula. As described above, a templates of the body 102 and/or gap filler 104 may be positioned over the image to determine a desired positioning and/or dimensions of the body 102 and the gap filler 104. Alternatively, a 3D template of the implant 100 may be generated using the image, to determine dimensions of the body 102 and the gap fillers 104.

Once the desired number of gap fillers 104, the dimensions and positioning thereof have been determined, the gap fillers 104 are formed on the exterior surface 106 of the body 102 via an additive machining process. Additive machining adds one or more gap fillers 104 to the body 102 layer by layer by melting the material to an exact geometry of the gap fillers 104 determined as described above. By additive machining, the implant 100 is formed without having to remove any material therefrom so that no material is wasted. In addition, additive machining permits the gap fillers 104 to be added to the body 102 including the openings 114. Thus, this technique allows a standard body 102 to be used with gap fillers 104 added thereto so that no material is wasted in adapting the implant 100 to the specific patient. In another embodiment, both the body 102 and the gap fillers 104 may be manufactured via additive machining in a single process such that the body 102 may also be customized to the dimensions determined as described above. Once the one or more gap fillers 104 have been added to the body 102, the implant 100 may be finished (e.g., ultrasonic cleaning, washing, high isostatic pressing, grinding, sterile packaging). The finished implant 100 is then fixed to the bone 10 using the bone fixation elements 112. The gap fillers 104 eliminate any gaps between the exterior surface 106 and portions of the bone 10 such that the bone fixation elements 112 are not required to carry all the load.

A method of forming an implant 200 according to the invention is substantially similar to the method described above for forming the implant 100, but further includes the step of determining whether openings or grooves exist in the bone to which the implant 200 is to be fixed. For example, openings or grooves may have been formed in the bone 10 during an earlier surgical procedure to accommodate portions of an implant, which was subsequently removed, or may naturally extend along a surface of the bone 10. The surgeon may determine from the imaged bone, whether such openings or grooves exist, and a positioning and dimensioning of the openings in the bone. In addition to forming the one or more gap fillers 204 on the body 202 via an additive machining process, one or more elongated elements 216 may also be formed on the exterior surface 206 of the body 202 via additive machining. The finished implant 200 is then fixed to the bone such that the elongated element 216 is inserted into the opening in the bone to provide increased stability thereof.

It will be apparent to those skilled in the art that various modifications and variations can be made in the structure and the methodology of the present invention, without departing from the spirit or the scope of the invention. Thus, it is intended that the present invention cover the modifications and variations of this invention provided that they come within the scope of the appended claims and their equivalents.

What is claimed is:

1. A patient-specific implant, comprising:
a body having a convex exterior surface configured to attach to a bone and a concave interior surface configured to receive a head portion of another bone, the body including a plurality of holes extending therethrough, the holes sized and shaped to receive a bone fixation element; and
a gap filler formed on the exterior surface of the body and having dimensions and a position corresponding to a gap between the exterior surface of the body and the bone to which the implant is to be attached.

2. The implant of claim 1, wherein the gap filler is formed of a porous material.

3. The implant of claim 1, wherein the gap filler is formed of a solid material.

4. The implant of claim 1, wherein the gap filler extends over at least one of the plurality of holes.

5. The implant of claim 4, wherein the gap filler includes an opening extending therethrough, the opening sized and shaped to correspond to the at least one of the plurality of holes.

6. The implant of claim 1, further comprising an elongated element formed on the exterior surface of the body, the elongated element sized and shaped to correspond to an opening in a bone to which the implant is to be attached.

7. The implant of claim 6, wherein the elongated element is cylindrical.

8. A method for forming a patient-specific implant, comprising:
imaging a bone to which the patient-specific implant is to be fixed;
determining based on the image dimensions and a position of a gap that would extend between an implant base and a surface of the bone if the implant base were implanted in a desired position and orientation on the bone, the implant base having a convex exterior surface configured to attach to the bone and a concave interior surface configured to receive a head portion of another bone and including a plurality of holes extending therethrough, each of the holes being sized and shaped to receive a bone fixation element therein; and
adding a gap filler to the exterior surface of the implant base via additive machining based on the determined dimensions and position of the gap so that the gap filler fills the gap.

9. The method of claim 8, further comprising positioning a template of a body of the implant over an image of the bone, in a desired orientation, the body having a convex exterior surface configured to attach to the bone and a concave interior surface configured to receive a head portion of another bone, the body including a plurality of holes extending therethrough, the holes sized and shaped to receive a bone fixation element.

10. The method of claim 9, further comprising positioning a template of the gap filler over the image of the bone to determine the dimensions and position of the gap.

11. The method of claim 8, further comprising obtaining a three dimensional template of the implant via the image of the bone to determine dimensions of the body and the gap filler.

12. The method of claim 8, wherein the gap filler extends over at least one of the plurality of holes.

13. The method of claim 12, further comprising forming an opening through the gap filler to correspond to the at least one of the plurality of holes.

14. The method of claim 8, wherein the gap filler is formed of a porous material.

15. The method of claim 8, wherein the gap filler is formed of a solid material.

16. The method of claim 8, further comprising identifying an opening formed through the bone during a prior surgical procedure.

17. The method of claim 16, further comprising determining dimensions and a position of the opening of the bone relative to the body.

18. The method of claim 17, further comprising adding an elongated element to the exterior surface of the body via additive machining based on the dimensions and position of the opening of the bone.

19. The method of claim 18, wherein the elongated element is cylindrical.

20. The method of claim 8, wherein the body and the gap filler are formed in a single additive machining process.
